(19) United States
(12) Patent Application Publication
(10) Pub. No.: US 2016/0141485 A1
(43) Pub. Date: May 19, 2016

(54) PIEZOELECTRIC ACTUATOR AND CAPSULE ENDOSCOPE INCLUDING THE SAME

(71) Applicant: SAMSUNG ELECTRO-MECHANICS CO., LTD., Suwon-Si (KR)

(72) Inventor: Dong Kyun LEE, Seoul (KR)

(73) Assignee: SAMSUNG ELECTRO-MECHANICS CO., LTD., Suwon-Si (KR)

(21) Appl. No.: 14/860,877

(22) Filed: Sep. 22, 2015

(30) Foreign Application Priority Data
Nov. 14, 2014 (KR) 10-2014-0158819

(51) Int. Cl. H01L 41/09; A61B 1/04; H01L 41/047

(52) U.S. Cl. CPC H01L 41/09; H01L 41/047; A61B 1/041

(57) ABSTRACT

A capsule endoscope includes a power supply operably coupled to a piezoelectric element. The power supply is configured to provide an alternating electric signal to the piezoelectric element. A housing defines a cavity therein to receive the piezoelectric element and the power supply. The piezoelectric element is configured to frictionally couple with an inner circumferential surface of the housing to impart movement.

### CROSS-REFERENCE TO RELATED APPLICATIONS

This application claims the benefit under 35 USC 119(a) of Korean Patent Application No. 10-2014-0158819, filed with the Korean Intellectual Property Office on Nov. 14, 2014, the entire disclosure of which is incorporated herein by reference for all purposes.

### BACKGROUND

1. Field

The following description relates to a piezoelectric actuator and a capsule endoscope having the same.

2. Description of Related Art

A piezoelectric actuator is an apparatus that is designed to convert vibrations of contraction and expansion occurring when electricity is supplied to a piezoelectric element to circular or linear motions by friction between a stator and a mover.

A capsule endoscope, which is a capsule type of endoscope, has a photographic apparatus installed in the capsule type of endoscope so as to take images of internal organs, allowing a subject to receive a medical examination of internal organs without pain or discomfort.

The capsule endoscope transmits photographed images to an outside while being moved by peristalsis of the internal organs, and the internal organs are examined based on the transmitted information.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect an a piezoelectric actuator is provided which has an inner circumferential electrode and an outer circumferential electrode formed, respectively, on an inner circumferential surface and an outer circumferential surface of a substantially annular piezoelectric element so as to allow vibrations of expansion and contraction to be generated by electric power supplied thereto, wherein the outer circumferential electrode is formed in plurality by being divided on the outer circumferential surface.

The piezoelectric actuator may further include a power supply configured to supply electric power in between the inner circumferential electrode and each of the plurality of outer circumferential electrodes.

The piezoelectric actuator may further include a controller configured to control electric signals being supplied to the outer circumferential electrodes to generate, respectively, different phase electric signals.

Another aspect provides a capsule endoscope including the piezoelectric actuator.

The capsule endoscope may include a housing covering the piezoelectric actuator and configured to allow vibrations of the piezoelectric element to be transferred by a friction at a portion in contact.

The capsule endoscope may further include a driving unit configured to allow an air current to be formed so as to be rotatable integrally with the housing.

The controller may be further configured to control electric signals having opposite directions from each other to be supplied, respectively, to a pair of the outer circumferential electrodes facing opposite to each other.

The controller may be configured to control an electric signal satisfying the following relationship to be successively supplied to each of the outer circumferential electrodes that are adjacent to one another:

phase difference of one circumferential electrode relative to a neighboring circumferential electrode=360°/(number of split electrodes formed on the outer circumferential electrode)

According to another general aspect, a capsule endoscope includes a piezoelectric element formed in a substantially annular shape and configured to have vibrations of expansion and contraction generated therein by electric power repeatedly supplied thereto. An inner circumferential electrode is formed on an inner circumferential surface of the piezoelectric element and an outer circumferential electrode is formed in plurality by being divided on an outer circumferential surface of the piezoelectric element. A power supply is configured to supply an electric signal between the inner circumferential electrode and each of the plurality of outer circumferential electrodes. A controller is configured to control electric signals being supplied to the outer circumferential electrodes in turn to generate, respectively, phase differences in the electric signal; and a housing is formed in an annular shape to cover outer circumferential surfaces of the outer circumferential electrodes, wherein an inner circumferential surface of the housing is brought into contact with a portion of the outer circumferential surface of the outer circumferential electrodes when the piezoelectric element receives the electric signal. The housing is configured to receive the vibrations of the piezoelectric element to be transferred by friction at the portion in contact.

The capsule endoscope may further include a driving unit coupled to the housing so as to be rotatable integrally with the housing and configured to allow a fluid current to be formed in a lengthwise direction of the housing by a rotation thereof.

The driving unit may further include a propeller having a plurality of blades about a rotation axis configured to convert a rotary motion of the housing to a linear motion of the capsule.

The controller may be further configured to control at least one of magnitude, direction or interval of the electric signal being applied to the outer circumferential electrode.

According to another general aspect, a capsule endoscope includes a deformable piezoelectric element; a power supply which is operably coupled to the piezoelectric element, and a housing. The power supply is configured to provide an alternating electric signal to the piezoelectric element. The housing defines a cavity therein to receive the piezoelectric element and the power supply. The piezoelectric element is configured to frictionally couple with an inner circumferential surface of the housing to impart movement.

The piezoelectric element may include a substantially annular member with a first pair of opposing circumferential portions.

The power supply may be configured to apply a complementary set of electric signals respectively to the first pair of opposing circumferential portions of the annular member.

The housing may further include a driving unit fixedly coupled thereto configured to displace a fluid responsive to movement of the housing.

The annular member may further include a second pair of opposing circumferential portions disposed transverse to the first circumferential portions

The power supply may further be configured to supply a first complementary set of signals to the first pair of opposing circumferential portions and a second complementary set of signals to the second pair of opposing circumferential portions.

The first pair of opposing circumferential portions may be supplied respectively with a sin and −sin signal and the second pair of opposing circumferential portions are supplied respectively with a cos and −cos signal.

The deformable piezoelectric element may include a number n of circumferentially disposed electrode portions, the power supply may be further configured to apply an electric signal to each of the circumferentially disposed electrode portions in sequence, with each application, the phase of the electrical signal being increased or decreased an amount substantially equal to 360/n.

The power supply may be further configured to concurrently supply an equal and opposite electric signal to a diametrically opposed circumferentially disposed electrode portion.

The housing may be deformable, and the piezoelectric element may be configured to deform the housing to increase a surface area of contact to increase a frictional engagement therewith.

Other features and aspects will be apparent from the following detailed description, the drawings, and the claims.

### BRIEF DESCRIPTION OF DRAWINGS

FIG. 1 shows an examplary capsule endoscope.

FIG. 2 shows an example of a piezoelectric element.

FIG. 3 is a cross-sectional view of the example piezoelectric element.

FIG. 4, FIG. 5 and FIG. 6 show how an example piezoelectric element vibrates when electricity is supplied to a piezoelectric actuator.

FIG. 7, FIG. 8, FIG. 9, FIG. 10, FIG. 11 and FIG. 12 illustrate various configurations of an exemplary capsule endoscope.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Hereinafter, certain examples of a piezoelectric actuator and a capsule endoscope having the same will be described in detail with reference to the accompanying drawings. In describing certain examples with reference to the accompanying drawings, any identical or corresponding elements will be assigned with same reference numerals, and their redundant description will not be provided.

When one element is described as being “coupled” or “connected” to another element, it shall be construed as not only being in physical contact with the other element but also possibly having a third element interposed therebetween and each of the one element and the other element being in contact with the third element.

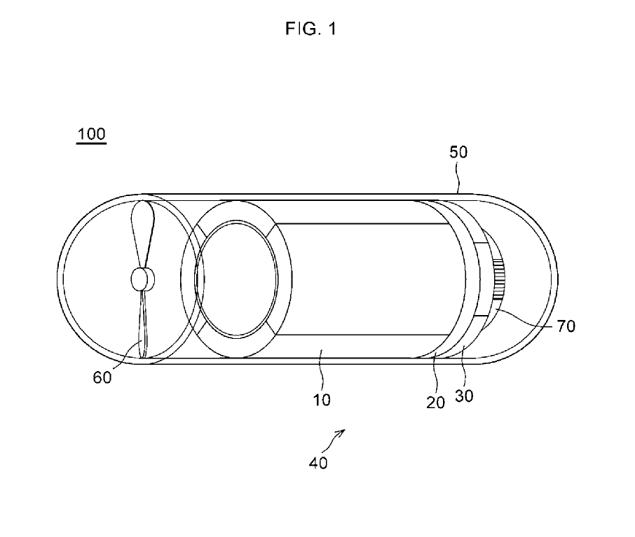

FIG. 1 shows an exemplary capsule endoscope.

Referring to FIG. 1, a capsule endoscope 100 includes a piezoelectric actuator 40, a housing 50, a driving unit 60 and a camera unit 70.

The piezoelectric actuator 40, which includes a piezoelectric element 10, a power supply 20 and a controller 30, uses the piezoelectric element 20 as a driving source to generate a mechanical movement.

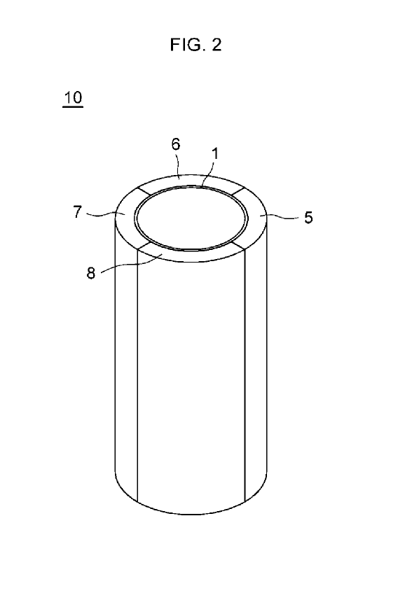

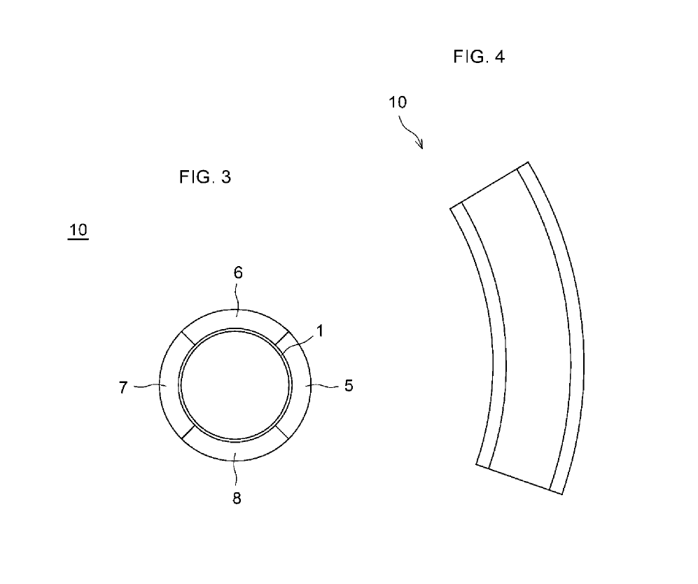

FIG. 2 shows the piezoelectric element 10, and FIG. 3 is a cross-sectional view of the piezoelectric element 10 in accordance with an embodiment of the present invention.

Referring to FIG. 2 and FIG. 3, the piezoelectric element 10 generates vibrations, using expanding and contracting properties, by electricity that is supplied repeatedly, alternatingly, continuously, and/or periodically.

The piezoelectric element 10 has an inner circumferential electrode 1 formed on an inner circumferential surface thereof and have outer circumferential electrodes 5, 6, 7, 8 divided and formed on an outer circumferential surface thereof.

By forming a plurality of outer circumferential electrodes 5, 6, 7, 8 that are divided from one another, different electrical signals may be supplied, respectively, to the plurality of outer circumferential electrodes 5, 6, 7, 8. In such manner, the plurality of outer circumferential electrodes 5, 6, 7, 8 may be selectively actuated to provide a range of movements.

By having the outer circumferential electrodes 5, 6, 7, 8 of the piezoelectric element 10 divided, it is possible to realize various displacements of the piezoelectric element 10, in addition to linear modification, because the displacement by which the piezoelectric element 10 is expanded or contracted may vary depending on the different types of electrical signals supplied to the outer circumferential electrodes 5, 6, 7, 8.

Although it is illustrated in FIG. 2 and FIG. 3 that the outer circumferential electrodes 5, 6, 7, 8 are divided into 4 for the convenience of description, the number of divided outer circumferential electrodes is not limited to what is illustrated herein, and the outer circumferential electrodes may be formed in any number of divisions.

The piezoelectric element 10 may be crystal, tourmaline, Rochelle salt, or any other suitable material which generates vibrations from mechanical deformation of expansion and contraction, and the piezoelectric element 10 may be formed in various shapes, for example, a circular cylinder, a polygonal cylinder, longitudinally extending strips, annular members, etc.

The piezoelectric element 10 may be formed by having the outer circumferential electrodes 5, 6, 7, 8 divided evenly. The plurality of outer circumferential electrodes 5, 6, 7, 8 formed on the outer circumferential surface of the piezoelectric element 10 may be formed to be symmetrical with one another.

The power supply 20 supplies electric signals between the inner circumferential electrode 1 and each of the outer circumferential electrodes 5, 6, 7, 8, and the controller 30 may control the electric signals supplied to the outer circumferential electrodes 5, 6, 7, 8 to generate phase differences that are different from one another.

The controller 30 may generate and apply opposite direction electric signals, respectively, to a pair of the outer circumferential electrodes 5, 6, 7, 8 that face each other from opposite sides.

For example, the outer circumferential electrode 5 may be supplied with an electric signal having a Sin phase, and the outer circumferential electrode 7, which faces the outer circumferential electrode 5 from the opposite side thereof, may be supplied with an electric signal having a −Sin phase.

Similarly, the outer circumferential electrode 6 may be supplied with an electric signal having a Cos phase, and the outer circumferential electrode 8 may be supplied with an electric signal having a −Cos phase.

In case a pair of facing electric signals are changed, electric signals supplied to their adjacent outer circumferential electrodes or the rest of the outer circumferential electrodes should not be changed in order to allow the electrodes of the piezoelectric element 10 to which the changed electric signals are supplied to be deformed only.

The controller 30 may control electric signals having a phase difference satisfying the following equation to be successively applied to adjacent outer circumferential electrodes 5, 6, 7, 8, respectively.

phase difference=360°/(number of split electrodes formed on the outer circumferential electrode) [Equation 1]

In the case where the controller 30 successively applies the electric signals to the outer circumferential electrodes 5, 6, 7, 8 so as to satisfy the above equation/relationship, the piezoelectric actuator 40 may rotate while generating vibrations, for example, facing vibrations.

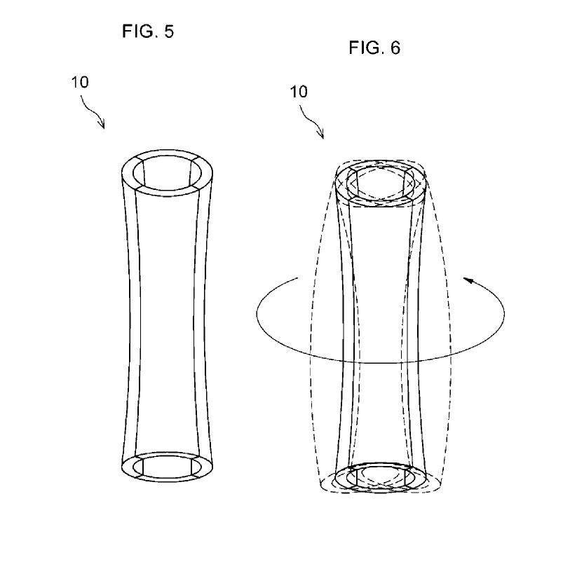

Facing vibrations refer to the piezoelectric actuator 40 being deformed in the lengthwise direction thereof, causing the piezoelectric actuator 40 to be bent and vibrate, as illustrated in FIG. 4.

The controller 30 may control at least one of the magnitude, direction and/or interval of electric signal being applied to the piezoelectric actuator 40. For example, the controller 30 or power supply 20 may modulate the magnitude of the electrical signal. The direction of the signal may be modulated; the interval or frequency of the signal may be suitably modulated; or combinations of the magnitude, direction, and interval may be modulated to induce suitable movement of the capsule.

The housing 50 is formed in an annular shape to cover outer circumferential surfaces of the outer circumferential electrodes 5, 6, 7, 8. The housing 50 is installed in such a way that an inner circumferential surface thereof is in contact with a portion of the outer circumferential surface of the outer circumference of the piezoelectric element 10, and may be vibrated by a friction or displacement of the piezoelectric element 10 at the contacted surface. Housing 50 may be a bio-compatible polymer or other suitable material such as titanium. Housing 50 may be rigid, or may be a deformable material which increases in surface-contact area with the piezoelectric element 10 as it protrudes outwardly. The inner circumferential surface of housing 50 may be frictionally engaged by the piezoelectric element 10. At least a portion of the housing 50 may be transparent to the visible spectrum and certain electromagnetic wavelengths employed for sensors, communication, guidance, and/or diagnostics. The housing 50 may include suitable fluid intake and outlet ports and or permeable or porous portions.

The driving unit 60 is coupled with the housing 50 so as to be rotatable integrally with the housing 50 and may be driven by a current in a lengthwise direction generated by a rotation of the housing 50.

The lengthwise or axial direction may be a direction in which the capsule endoscope 100 moves forward or backward, and the direction of the capsule endoscope 100 may be changed forward or backward when the rotation of the driving unit 60 is changed.

The driving unit 60 may propel the capsule endoscope 100 by converting a torque of the piezoelectric actuator 40 to a propulsive force. While the Figures show the housing 50 extending and encircling the driving unit, there may be suitable holes or openings provided to allow a bodily fluid such as air, water, blood, or the like to flow through the driving unit 60 for propulsive, diagnostic, and/or treatment purposes.

The driving unit 60 is integrally coupled with the housing 50 to provide the propulsive force to the capsule endoscope 100 while being driven by a rotary current generated by vibrations of the piezoelectric actuator 40.

The driving unit 60 may include a rotation axis and a propeller having a plurality of blades about the rotation axis so as to convert a rotary motion of the housing 50 to a linear motion.

The capsule endoscope 100 may also include the camera unit 70 for recording images of internal organs and may further include a communication unit, for transmitting information recorded by the endoscope 100 wirelessly, and various sensors. Additionally, light and/or sound transceivers or transducers may be included for diagnostic, communication, and treatment purposes.

FIG. 4, FIG. 5 and FIG. 6 show how the piezoelectric element 10 vibrates when electricity is supplied to the piezoelectric actuator.

Hereinafter, the principle of how the piezoelectric element 10 is vibrated will be described with reference to FIG. 4 to FIG. 6.

The piezoelectric element 10 may be vibrated by supplying electric signals having phase differences to the plurality of outer circumferential electrodes 5, 6, 7, 8 formed on the outer circumferential surface of the piezoelectric element 10.

For example, among the plurality of outer circumferential electrodes 5, 6, 7, 8, the outer circumferential electrode 5 may be supplied with an electric signal of sin waves, and the outer circumferential electrode 7, which is symmetrically opposite to the outer circumferential electrode 5, may be supplied with an electric signal of −sin waves, to allow the outer circumferential electrode 5 to expand in the direction in which the electric signal of the sin waves is supplied and to allow the outer circumferential electrode 7 to contract in the direction in which the electric signal of the −sin waves is supplied. In such manner, complementary signals may be applied to respectively opposing electrodes.

In the case where the outer circumferential electrode 5 and the outer circumferential electrode 7 expand and contract, respectively, the electric signals supplied to the outer circumferential electrode 6 and the outer circumferential electrode 8, which are adjacent to the outer circumferential electrode 5 and outer circumferential electrode 7, are controlled to be unchanged so that the facing vibrations occur in the direction in which the outer circumferential electrode 5 expands.

After the opposite-direction electric signals are supplied, respectively, to the outer circumferential electrode 5 and the outer circumferential electrode 7, the outer circumferential electrode 6 may be supplied with an electric signal having cos waves, and the outer circumferential electrode 8 may be supplied with an electric signal having −cos waves, resulting in a contracting deformation of the outer circumferential electrode 8, which is symmetrically opposite to the outer circumferential electrode 6 supplied with the electric signal of cos waves. It should be understood that the outer circumferential electrodes are not limited to sin, −sin, cos, −cos signals, but can be any set of substantially complementary or opposite signals. While outer circumferential electrodes 5. 6, 7 and 8 have been described with reference to cos and −cos signals, any suitable signals may be employed.

By repeatedly supplying electric signals having 90-degree phase differences to adjacent outer circumferential electrodes 5, 6, 7, 8 successively, the piezoelectric actuator 40 may have the facing vibrations occurred repeatedly.

By transferring electric signals to the outer circumferential electrode 5, the outer circumferential electrode 6, the outer circumferential electrode 7 and the outer circumferential electrode 8 successively, the piezoelectric actuator 40 may be induced to have facing vibrations and rotary motions successively.

When the piezoelectric actuator 40 has the facing vibrations and rotary motions successively, the piezoelectric actuator 40 vibrates as if a hula hoop is rotated.

When the piezoelectric actuator 40 vibrates as described above, only a portion of the inner circumferential surface of the housing 50 makes contact with the piezoelectric actuator 40 and generates friction.

The reason why the piezoelectric actuator 40 and the inner circumferential surface of the housing 50 make contact with each other only at a portion thereof to generate friction is that only a portion of the piezoelectric actuator 40 bulged by the facing vibrations makes contact with the inner circumferential surface of the housing 50.

A direction in which the contacted surface, where the friction occurs, is changed coincides with the direction of rotation of the piezoelectric actuator 40 by the facing vibrations, thereby allowing the housing 50 to be rotated.

When the housing 50 is rotated, a rotating current is formed within the housing 50, and the generated rotating current drives the driving unit 60, which is coupled with the housing 50 so as to be integrally rotated with the housing 50.

In the same way as a propeller is driven, the driving unit 60 creates more flow of fluid such as air as the propeller is rotated, and the capsule endoscope 100 may be driven by a difference of air speed and a difference of air pressure formed on one side of the propeller.

In order to control the piezoelectric actuator 40 to have the facing vibrations, the phase difference may be formed to satisfy the following equation.

phase difference=360°/(number of split electrodes formed on the outer circumferential electrode) [Equation 1]

For example, if there are four split electrodes formed on the outer circumferential electrodes 5, 6, 7, 8, the electric signals may be controlled in such a way that the phase difference between the outer circumferential electrode 5, the outer circumferential electrode 6, the outer circumferential electrode 7 and the outer circumferential electrode 8, which are adjacent to one another, is 90 degrees.

Moreover, if there are six split electrodes formed on the outer circumferential electrodes 5, 6, 7, 8, the electric signals may be controlled in such a way that the phase difference between the adjacent outer circumferential electrodes is 60 degrees.

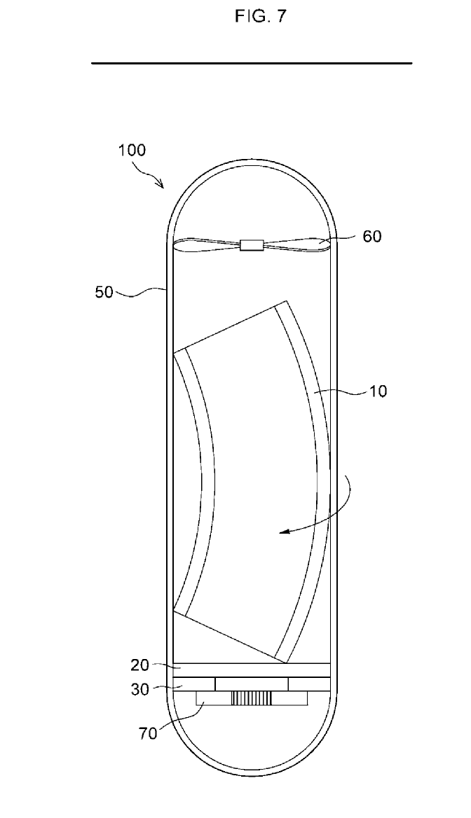

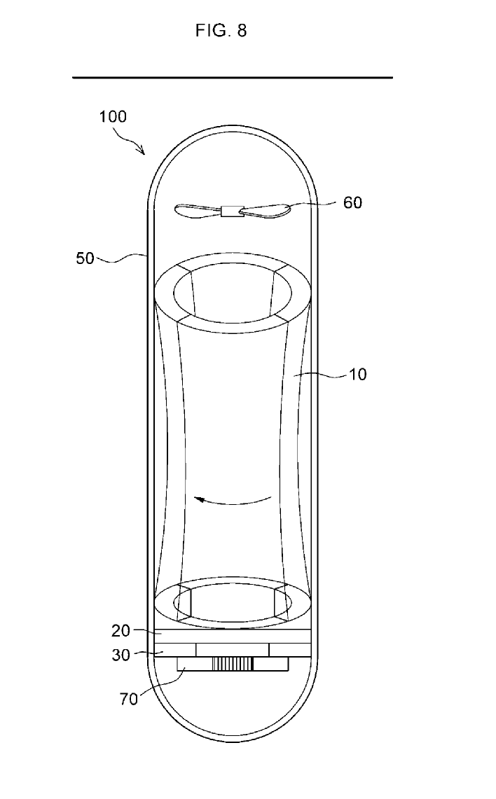

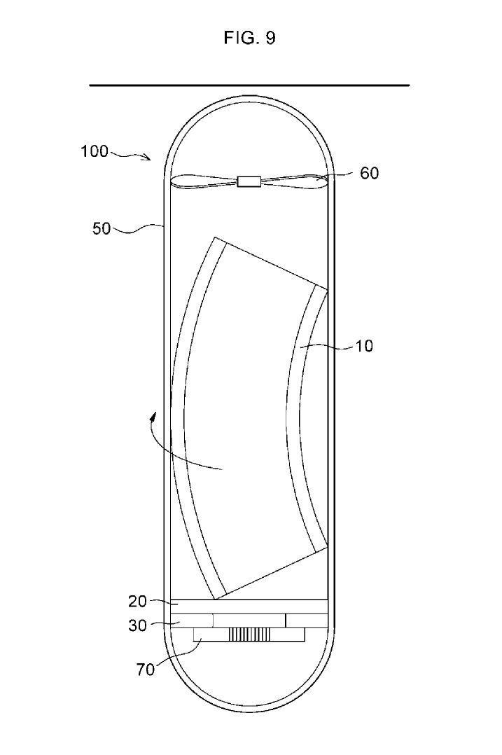

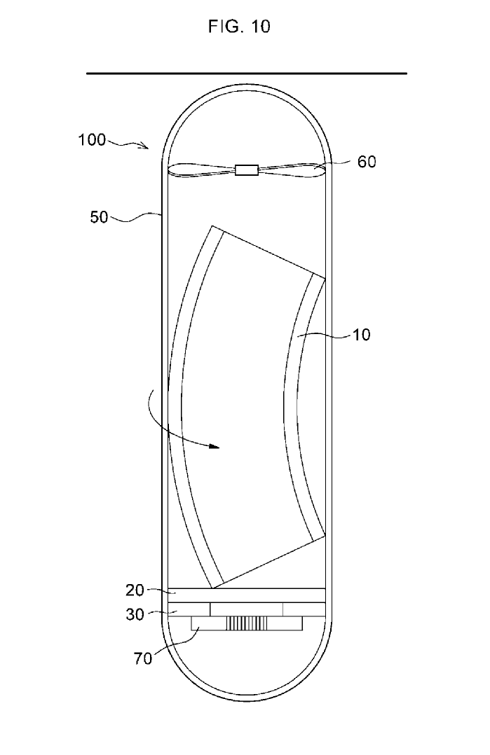

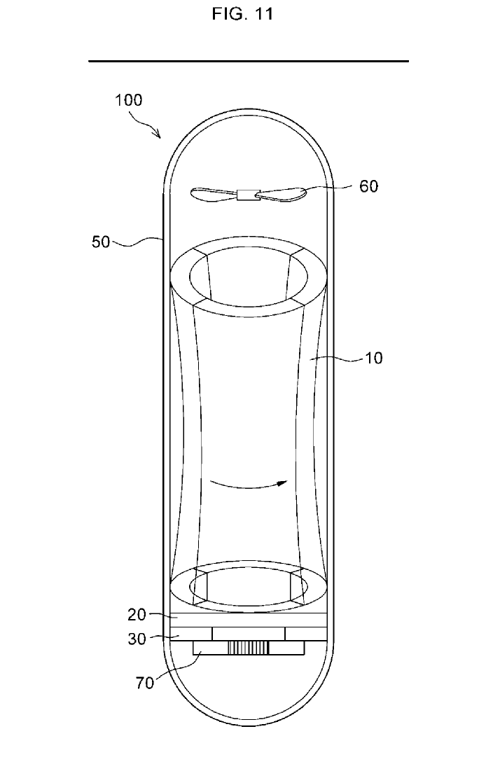

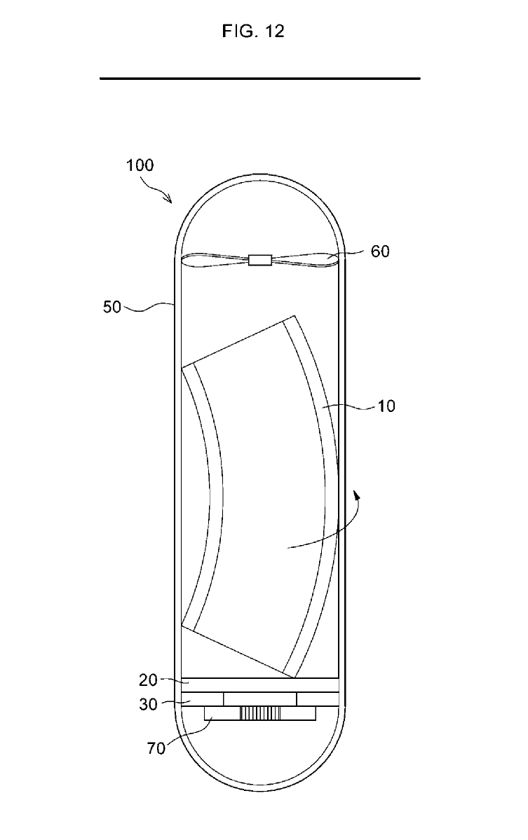

FIG. 7, FIG. 8, FIG. 9, FIG. 10, FIG. 11 and FIG. 12 show how the capsule endoscope 100 is driven forward or backward.

Referring to FIG. 7 to FIG. 12, it can be seen that it is possible to control the position of the capsule endoscope 100, through the control of electric signals, to allow the capsule endoscope 100 to move toward or away from a reference line.

Although a conventional capsule endoscope may be displaced by peristalsis of the internal organs, it is possible to allow the capsule endoscope 100 to be moved forward or backward by the control of electric signals.

Moreover, the capsule endoscope 100 may be kept at a fixed position by cutting off the electric signals by use of the off-power holding property of the piezoelectric actuator 40.

This property of the piezoelectric actuator 40 allows the capsule endoscope 100 to be controlled more easily because no other electric signal is required to stabilize the position of the capsule endoscope 100. In some configurations, a fixed position may be maintained by adaptively generating signals of substantially equal magnitude and opposite direction of the normal displacement due to peristalsis.

Moreover, a driving speed of the capsule endoscope 100 may be controlled by the magnitude of the electric signal supplied to the piezoelectric actuator 40, and a driving angle of the capsule endoscope 100 may be controlled by measuring the number of rotations of the piezoelectric actuator 40 for a given duration of supply of the electric signal to the piezoelectric actuator 40.

The capsule endoscope 100 may save the consumption of electric power because the piezoelectric actuator 40 is used as the source of driving power.

Moreover, the piezoelectric actuator 40 generates neither electromagnetic waves nor noise, allowing a subject to receive a medical examination with less discomfort.

Furthermore, when the piezoelectric actuator 40 is utilized as the driving source of the capsule endoscope 100, the medical examination may be performed faster and more precisely, owing to the higher energy density, faster response speed and higher positional precision of the piezoelectric actuator 40.

The controller 20 and other components illustrated in FIGS. 1-12 that perform the operations described herein with respect to FIGS. 6-12 are implemented by hardware components. Examples of hardware components include controllers, sensors, generators, drivers, and any other electronic components known to one of ordinary skill in the art. In one example, the hardware components are implemented by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein with respect to FIGS. 6-12.

A computing system or a computer may include a microprocessor that is electrically connected to a bus, a user interface, and a memory controller, and may further include a flash memory device. The flash memory device may store N-bit data via the memory controller. The N-bit data may be data that has been processed and/or is to be processed by the microprocessor, and N may be an integer equal to or greater than 1. If the computing system or computer is a mobile device, a battery may be provided to supply power to operate the computing system or computer. It will be apparent to one of ordinary skill in the art that the computing system or computer may further include an application chipset, a camera image processor, a mobile Dynamic Random Access Memory (DRAM), or any other device known to one of ordinary skill in the art as being suitable for inclusion in a computing system or computer. The memory controller and the flash memory device may constitute a solid-state drive or disk (SSD) that uses non-volatile memory to store data.

The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 6-12 that perform the operations described herein are performed by a processor or a computer as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on methods illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files such as pictures, diagnostic data, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A piezoelectric actuator comprising:
a piezoelectric element formed in a substantially annular shape and configured to have vibrations of expansion and contraction generated therein by electric power repeatedly supplied thereto;
an inner circumferential electrode formed on an inner circumferential surface of the piezoelectric element; and
an outer circumferential electrode formed in plurality by being divided on an outer circumferential surface of the piezoelectric element.

2. The piezoelectric actuator of claim 1, further comprising:
a power supply configured to supply electric power between the inner circumferential electrode and each of the plurality of outer circumferential electrodes; and
a controller configured to control electric signals being supplied to the outer circumferential electrodes to generate, respectively, signals of different phases.

3. The piezoelectric actuator of claim 2, wherein the controller is configured to control electric signals having opposite directions from each other to be supplied, respectively, to a pair of the outer circumferential electrodes facing opposite to each other.

4. The piezoelectric actuator of claim 2, wherein the controller is configured to control an electric signal satisfying the following relationship to be successively supplied to each of the outer circumferential electrodes that are adjacent to one another:
phase difference of one circumferential electrode relative to a neighboring circumferential electrode=360°/(number of split electrodes formed on the outer circumferential electrode)

5. A capsule endoscope comprising:
a piezoelectric element formed in a substantially annular shape and configured to have vibrations of expansion and contraction generated therein by electric power repeatedly supplied thereto;
an inner circumferential electrode formed on an inner circumferential surface of the piezoelectric element;
an outer circumferential electrode formed in plurality by being divided on an outer circumferential surface of the piezoelectric element;
a power supply configured to supply an electric signal between the inner circumferential electrode and each of the plurality of outer circumferential electrodes;
a controller configured to control electric signals being supplied to the outer circumferential electrodes in turn to generate, respectively, phase differences in the electric signal; and
a housing formed in an annular shape to cover outer circumferential surfaces of the outer circumferential electrodes, wherein an inner circumferential surface of the housing is brought into contact with a portion of the outer circumferential surface of the outer circumferential electrodes when the piezoelectric element receives the electric signal, and configured to receive the vibrations of the piezoelectric element to be transferred by a friction at the portion in contact.

6. The capsule endoscope of claim 5, wherein the controller is configured to control electric signals having opposite directions from each other to be supplied, respectively, to a pair of the outer circumferential electrodes facing opposite to each other.

7. The capsule endoscope of claim 5, wherein the controller is configured to control an electric signal satisfying the following relationship to be successively supplied to each of the outer circumferential electrodes that are adjacent to one another:
phase difference=360°/(number of split electrodes formed on the outer circumferential electrode)

8. The capsule endoscope of claim 5, further comprising a driving unit coupled to the housing so as to be rotatable integrally with the housing and configured to allow a fluid current to be formed in a lengthwise direction of the housing by a rotation thereof.

9. The capsule endoscope of claim 8, wherein the driving unit comprises a propeller having a plurality of blades about a rotation axis configured to convert a rotary motion of the housing to a linear motion of the capsule.

10. The capsule endoscope of claim 5, wherein the controller is configured to control at least one of magnitude, direction or interval of the electric signal being applied to the the piezoelectric element.

11. A capsule endoscope comprising:
a power supply operably coupled to a piezoelectric element, the power supply configured to provide an alternating electric signal to the piezoelectric element; and,
a housing defining a cavity therein to receive the piezoelectric element and the power supply, wherein the piezoelectric element is configured to frictionally couple with an inner circumferential surface of the housing to impart movement.

12. The capsule endoscope of claim 11, wherein the piezoelectric element comprises a substantially annular member with a first pair of opposing circumferential portions.

13. The capsule endoscope of claim 12, wherein the power supply is configured to apply a complementary set of electric signals respectively to the first pair of opposing circumferential portions of the annular member.

14. The capsule endoscope of claim 13, wherein the housing further comprises a driving unit fixedly coupled thereto configured to displace a fluid responsive to movement of the housing.

15. The capsule endoscope of claim 14, wherein the annular member further comprises a second pair of opposing circumferential portions disposed transverse to the first circumferential portions.

16. The capsule endoscope of claim 15, wherein the power supply is further configured to supply a first complementary set of signals to the first pair of opposing circumferential portions and a second complementary set of signals to the second pair of opposing circumferential portions.

17. The capsule endoscope of claim 15, wherein the first pair of opposing circumferential portions are supplied respectively with a sin and −sin signal and the second pair of opposing circumferential portions are supplied respectively with a cos and −cos signal.

18. The capsule endoscope of claim 11, wherein the deformable piezoelectric element comprises a number n of circumferentially disposed electrode portions, the power supply is further configured to apply an electric signal to each of the circumferentially disposed electrode portions in sequence, with each application, the phase of the electrical signal being increased or decreased an amount substantially equal to 360/n.

19. The capsule endoscope of claim 18, wherein the power supply is further configured to concurrently supply an equal and opposite electric signal to a diametrically opposed circumferentially disposed electrode portion.

20. The capsule endoscope of claim 11, wherein the housing is deformable, and the piezoelectric element is configured to deform the housing to increase a surface area of contact to increase a frictional engagement therewith.
